(19) United States
(12) Patent Application Publication
(10) Pub. No.: US 2023/0101623 A1
(43) Pub. Date: Mar. 30, 2023

(54) ELECTROSURGICAL SYSTEM WITH OPTICAL SENSOR ELECTRONICS

(71) Applicant: Cilag GmbH International, Zug (CH)

(72) Inventors: Chad P. Boudreaux, Cincinnati, OH (US); Thomas J. Watson, Over (GB)

(21) Appl. No.: 17/490,045

(22) Filed: Sep. 30, 2021

(51) Int. Cl. A61B 18/14; A61B 18/12; A61B 90/30

(52) U.S. Cl. CPC A61B 18/1445; A61B 18/1206; A61B 90/30; A61B 2018/00172

(57) ABSTRACT

A surgical system includes a surgical instrument, including a shaft assembly having a distal end and an end effector at the distal end of the shaft assembly. The end effector includes a first jaw, a second jaw movably coupled relative to the first jaw for clamping tissue therebetween, and an optical sensor for detecting the tissue. The surgical system also includes a generator configured to supply a therapeutic energy to the first jaw or the second jaw, and a pass-through device configured to be connected between the surgical instrument and the generator. The pass-through device includes a therapeutic energy connector configured to operatively couple the generator to the surgical instrument for transmitting the therapeutic energy from the generator to the first jaw or the second jaw, and at least one optical component configured to transmit light to the optical sensor and to receive light from the optical sensor.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio. Further examples of such devices and related concepts are disclosed in U.S. Pat. No. 6,500,176 entitled “Electrosurgical Systems and Techniques for Sealing Tissue,” issued Dec. 31, 2002, the disclosure of which is incorporated by reference herein, in its entirety; U.S. Pat. No. 8,939,974, entitled “Surgical Instrument Comprising First and Second Drive Systems Actuatable by a Common Trigger Mechanism,” issued Jan. 27, 2015, the disclosure of which is incorporated by reference herein, in its entirety; U.S. Pat. No. 8,888,809, entitled “Surgical Instrument with Jaw Member,” issued Nov. 18, 2014, the disclosure of which is incorporated by reference herein, in its entirety; U.S. Pat. No. 9,161,803, entitled “Motor Driven Electrosurgical Device with Mechanical and Electrical Feedback,” issued Oct. 20, 2015, the disclosure of which is incorporated by reference herein, in its entirety; U.S. Pat. No. 9,877,720, entitled “Control Features for Articulating Surgical Device,” issued Jan. 30, 2018, the disclosure of which is incorporated by reference herein, in its entirety; U.S. Pat. No. 9,545,253, entitled “Surgical Instrument with Contained Dual Helix Actuator Assembly,” issued Jan. 17, 2017, the disclosure of which is incorporated by reference herein, in its entirety; and U.S. Pat. No. 9,526,565, entitled “Electrosurgical Devices,” issued Dec. 27, 2016, the disclosure of which is incorporated by reference herein, in its entirety.

Some electrosurgical instruments include an end effector with at least one compliant feature. Examples of such instruments are described in U.S. Pat. No. 9,149,325, entitled “End Effector with Compliant Clamping Jaw,” issued Oct. 6, 2015, the disclosure of which is incorporated by reference herein, in its entirety; and U.S. Pat. No. 9,877,782, entitled “Electrosurgical Instrument End Effector with Compliant Electrode,” issued Jan. 30, 2018, the disclosure of which is incorporated by reference herein, in its entirety.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a first exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4A depicts a side elevational view of a handle assembly of the electrosurgical instrument of FIG. 1, where the end effector is in an open and unfired state, where a portion of the handle assembly is omitted for purposes of clarity;

FIG. 4B depicts a side elevational view of the handle assembly of FIG. 4A, where the end effector is in a closed and unfired state, where a portion of the handle assembly is omitted for purposes of clarity;

FIG. 4C depicts a side elevational view of the handle assembly of FIG. 4A, where the end effector is in a closed and fired state, where a portion of the handle assembly is omitted for purposes of clarity;

FIG. 5A depicts a cross-sectional side view of the end effector of FIG. 2, where the end effector is in the open and unfired state, taken along line 5-5 of FIG. 2;

FIG. 5B depicts a cross-sectional side view of the end effector of FIG. 2, where the end effector is in the closed and unfired state, taken along line 5-5 of FIG. 2;

FIG. 5C depicts a cross-sectional side view of the end effector of FIG. 2, where the end effector is in the closed and fired state, taken along line 5-5 of FIG. 2;

FIG. 6 depicts a perspective view of an exemplary electrosurgical system including a second exemplary electrosurgical instrument operatively coupled to an RF generator via a cable and an optical detection-enabling pass-through box;

FIG. 7 depicts a schematic view of a portion of the electrosurgical system of FIG. 6, showing various electronic components of the RF generator, pass-through box, and cable operatively coupled to each other;

FIG. 8 depicts a perspective view of a proximal cable plug of the cable of FIG. 6, showing a key portion of the proximal cable plug configured to prevent the cable from being directly coupled to the RF generator in the absence of the pass-through box;

FIG. 9 depicts a perspective view of another exemplary electrosurgical system including a third exemplary electrosurgical instrument operatively coupled to an RF generator and to a spectrometer via a cable and an optical detection-enabling pass-through box;

FIG. 10 depicts a schematic view of a portion of the electrosurgical system of FIG. 9, showing various electronic components of the RF generator, spectrometer, pass-through box, and cable operatively coupled to each other;

FIG. 11 depicts a perspective view of yet another exemplary electrosurgical system including a fourth exemplary electrosurgical instrument operatively coupled to an RF generator via a cable having an optical detection-enabling pass-through distal plug;

FIG. 12 depicts a schematic view of a portion of the electrosurgical system of FIG. 6, showing various electronic components of the RF generator, cable, and electrosurgical instrument operatively coupled to each other; and

FIG. 13 depicts a schematic view of a fifth exemplary electrosurgical instrument including an optical detection-enabling pass-through proximal body and a distal body removably coupled to each other, showing various electronic components of the electrosurgical instrument operatively coupled to each other.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

### I. Example of Electrosurgical Instrument

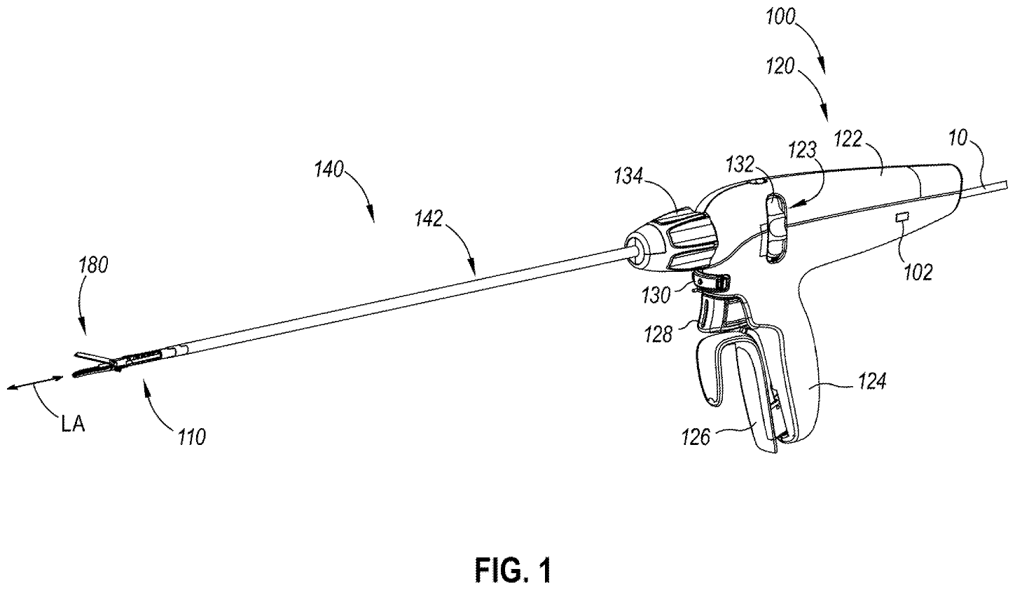

FIGS. 1-3C show a first exemplary electrosurgical instrument (100). As best seen in FIG. 1, electrosurgical instrument (100) includes a handle assembly (120), a shaft assembly (140), an articulation assembly (110), and an end effector (180). As will be described in greater detail below, end effector (180) of electrosurgical instrument (100) is operable to grasp, cut, and seal or weld tissue (e.g., a blood vessel, etc.). In this example, end effector (180) is configured to seal or weld tissue by applying bipolar radio frequency (RF) energy to tissue. However, it should be understood electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) is electrically coupled to a power source (not shown) via power cable (10).

The power source may be configured to provide all or some of the electrical power requirements for use of electrosurgical instrument (100). Any suitable power source may be used as would be apparent to one skilled in the art in view of the teachings herein. By way of example only, the power source may comprise a GEN04 or GEN11 sold by Ethicon Endo-Surgery, Inc. of Cincinnati, Ohio. In addition, or in the alternative, the power source may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While in the current example, electrosurgical instrument (100) is coupled to a power source via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) is configured to be grasped by an operator with one hand, such that an operator may control and manipulate electrosurgical instrument (100) with a single hand. Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) from the longitudinal axis (LA) defined by shaft assembly (140).

Handle assembly (120) includes a control unit (102) housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrode surfaces (194, 196) of jaws (182, 184), respectively.

Body (122) of handle assembly (120) defines an opening (123) in which a portion of articulation control (132) protrudes from. Articulation control (132) is rotatably disposed within body (122) such that an operator may rotate the portion of articulation control (132) protruding from opening (123) to rotate the portion of articulation control (132) located within body (122). Rotation of articulation control (132) relative to body (122) is configured to bend articulation section (110) in order to drive deflection of end effector (180) from the longitudinal axis (LA) defined by shaft assembly (140). Articulation control (132) and articulation section (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

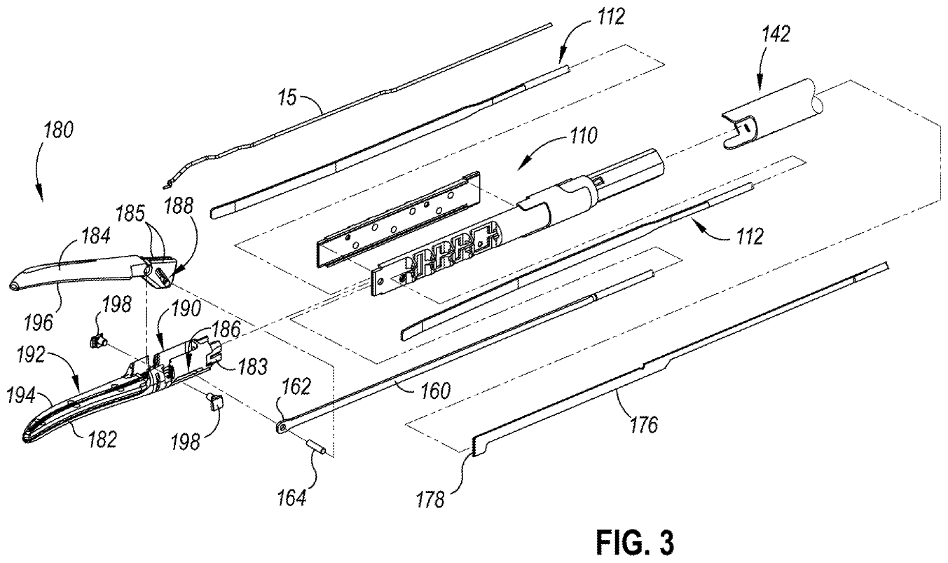

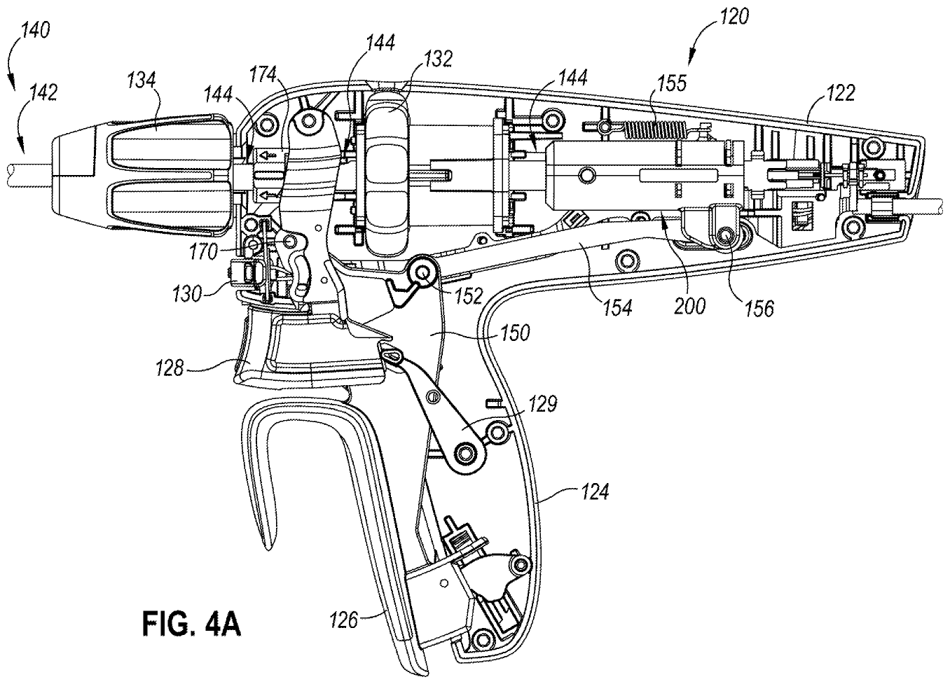

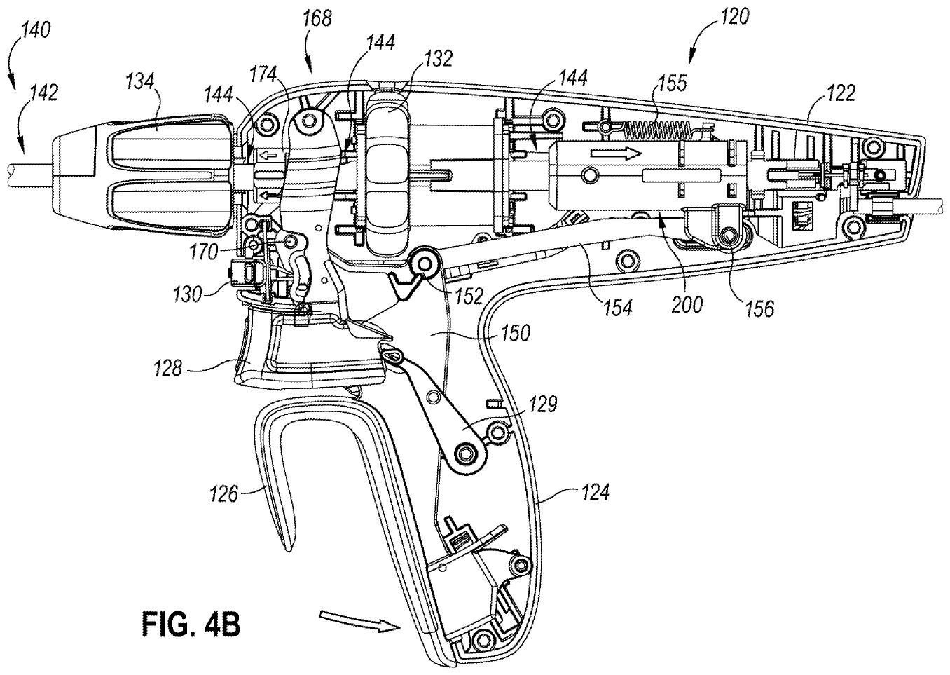

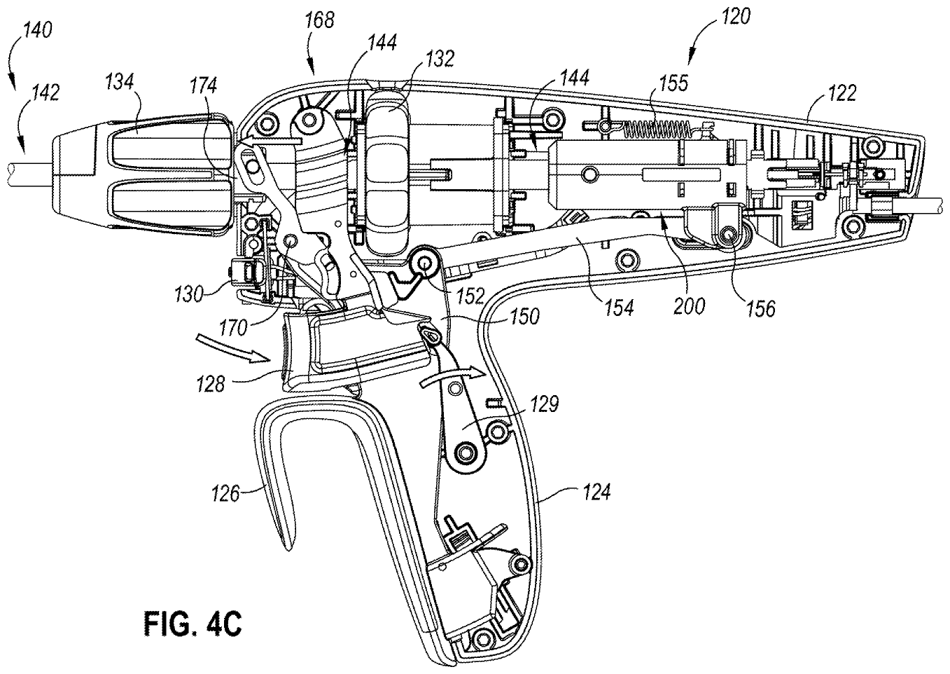

Shaft assembly (140) includes distal portion (142) extending distally from handle assembly (120), and a proximal portion (144) (see FIGS. 4A-4B) housed within the confines of body (122) of handle assembly (120). As best shown in FIG. 3, shaft assembly (140) houses a jaw closure connector (160) that couples jaw closure trigger (126) with end effector (180). Additionally, shaft assembly (140) houses a portion of knife member extending between distal cutting edge (178) and knife trigger (128). Shaft assembly (140) also houses actuating members (112) that couple articulation assembly (110) with articulation control (132); as well as an electrical connecter (15) that operatively couples electrode surfaces (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180); and activation button (130) is configured to activate electrode surface (194, 196).

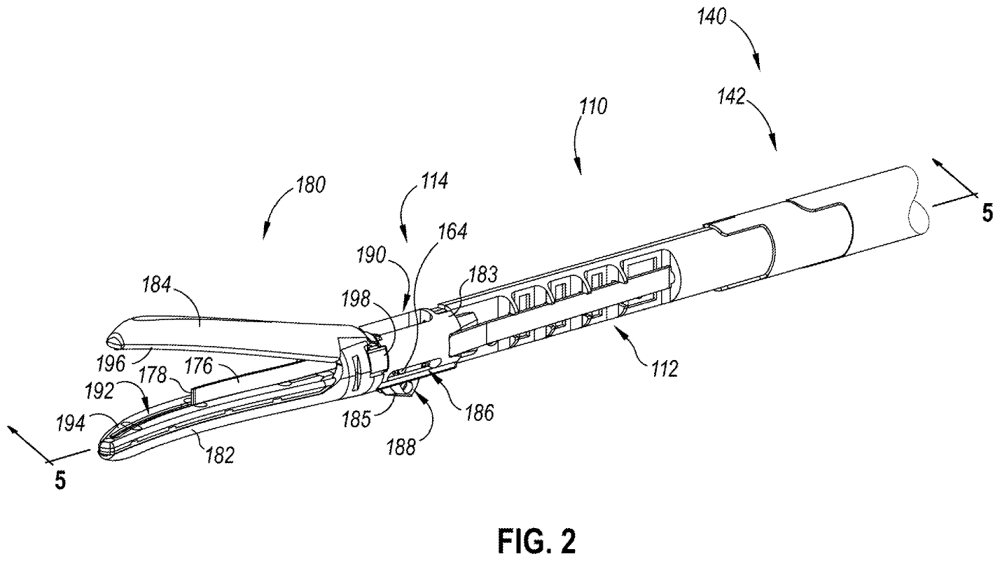

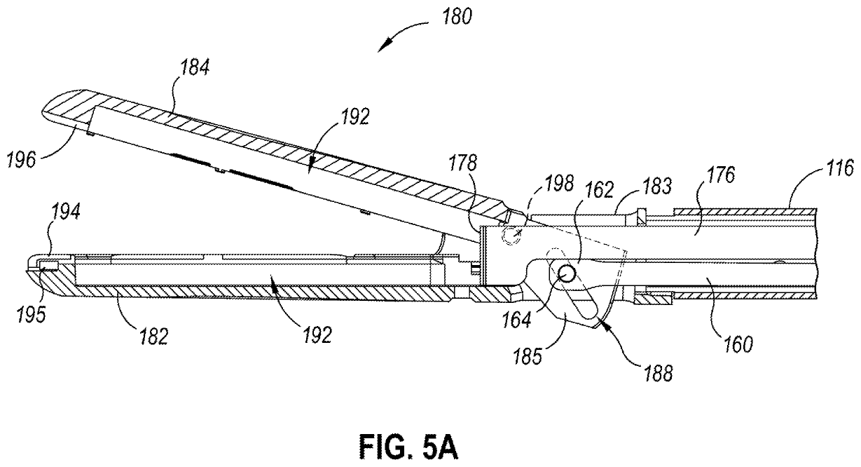

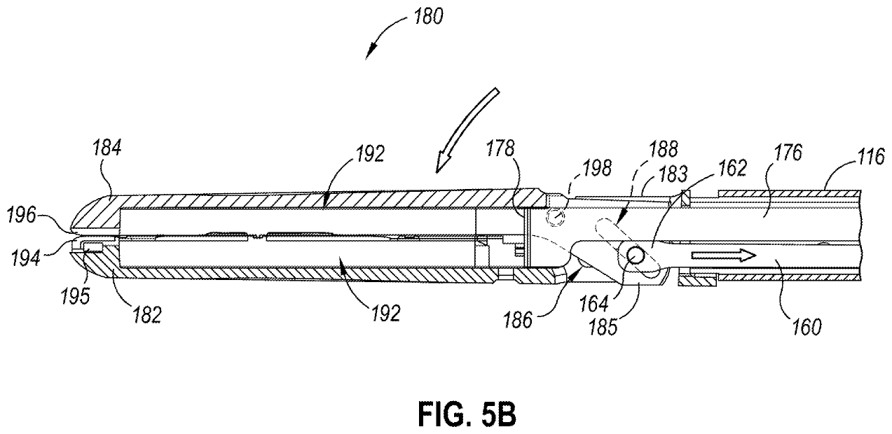

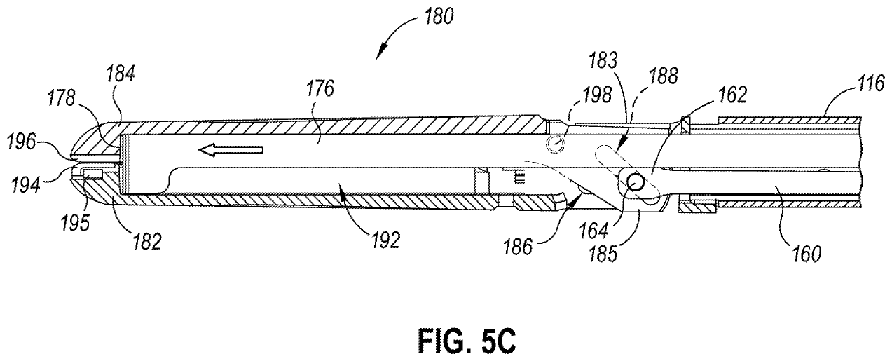

As best seen in FIGS. 2-3, end effector (180) includes lower jaw (182) pivotally coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connecter (160), and pin (164). Slots (186, 188) each slidably receive pin (164), which is attached to a distal coupling portion (162) of jaw closure connector (160). Additionally, as best seen in FIGS. 5A-5C, lower jaw (182) includes a force sensor (195) located at a distal tip of lower jaw (182). Force sensor (195) may be in communication with control unit (102). Force sensor (195) may be configured to measure the closure force generated by pivoting jaws (182, 184) into a closed configuration in accordance with the description herein. Additionally, force sensor (195) may communicate this data to control unit (102). Any suitable components may be used for force sensor (195) as would be apparent to one skilled in art in view of the teachings herein. For example, force sensor (195) may take the form of a strain gauge.

While in the current example, a force sensor (195) is incorporated into instrument (100) and is in communication with control unit (102), any other suitable sensors or feedback mechanisms may be additionally or alternatively incorporated into instrument (100) while in communication with control unit (102) as would be apparent to one skilled in the art in view of the teachings herein. For instance, an articulation sensor or feedback mechanism may be incorporated into instrument (100), where the articulation sensor communicates signals to control unit (102) indicative of the degree end effector (180) is deflected from the longitudinal axis (LA) by articulation control (132) and articulation section (110).

As will be described in greater detail below, jaw closure connector (160) is operable to translate within central channel (190) of lower jaw (182). Translation of jaw closure connector (160) drives pin (164). As will also be described in greater detail below, with pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

The term “pivot” does not necessarily require rotation about a fixed axis and may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) also define a knife pathway (192). Knife pathway (192) is configured to slidably receive knife member (176), such that knife member (176) may be retracted (as shown in FIGS. 5A-5B), and advanced (as shown in FIG. 5C), to cut tissue captured between jaws (182, 184). Lower jaw (182) and upper jaw (184) each comprise a respective electrode surface (194, 196). The power source may provide RF energy to electrode surfaces (194, 196) via electrical coupling (15) that extends through handle assembly (120), shaft assembly (140), articulation assembly (110), and electrically couples with one or both of electrode surfaces (194, 196). Electrical coupling (15) may selectively activate electrode surfaces (194, 196) in response to an operator pressing activation button (130). In some instances, control unit (102) may couple electrical coupling (15) with activation button (130), such that control unit (102) activates electrode surfaces (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc.

FIGS. 4A-5C show an exemplary use of instrument (100) for end effector (180) to grasp, cut, and seal/weld tissue. As described above, and as shown between FIGS. 4A-4B and 5A-5B, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

Handle assembly (120) further includes a yoke assembly (200) that is slidably coupled along proximal portion (144) of shaft assembly (140). Yoke assembly (200) is operatively coupled with jaw closure connector (160) such that translation of yoke assembly (200) relative to proximal portion (144) of shaft assembly (140) translates jaw closure connector (160) relative to shaft assembly (140).

As best seen in FIGS. 4A-4C, yoke assembly (200) is coupled to a body (150) of jaw closure trigger (126) via a link (154). Link (154) is pivotally coupled with yoke assembly (200) via pin (156); while link (154) is also pivotally coupled with body (150) of jaw closure trigger (126) via pin (152). Additionally, jaw closure trigger (126) is pivotally coupled with body (122) of handle assembly (120) via pin (170). Therefore, as shown between FIGS. 4A-4B, an operator may pull jaw closure trigger (126) toward pistol grip (124), thereby rotating jaw closure trigger (126) about pin (170). Rotation of jaw closure trigger (126) leads to rotation of link (154) about both pins (152, 156), which in turn drives yoke assembly (200) in the proximal direction along proximal portion (144) of shaft assembly (140).

As described above, jaw closure connector (160) extends within shaft assembly (140), articulation section (110), and central channel (190) of lower jaw (182). As also mentioned above, jaw closure connector (160) is attached to pin (164). Therefore, as seen between FIGS. 5A-5B, proximal translation of yoke assembly (200) leads to proximal translation of pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

As best seen in FIGS. 4A-4C, yoke assembly (200) is also coupled with a bias spring (155). Bias spring (155) is also coupled to a portion of body (122), such that bias spring (155) biases yoke assembly (200) to the position shown in FIG. 4A (associated with the open configuration of end effector (180) as shown in FIG. 5A). Therefore, if an operator releases jaw closure trigger (126), bias spring (155) will translate yoke assembly (200) to the position shown in FIG. 4A, thereby opening jaws (182, 184) of end effector (180).

As described above, and as shown between FIGS. 4B-4C and 5B-5C, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body (174) that is slidably coupled along proximal portion (144) of shaft assembly (140). Knife coupling body (174) is coupled with knife member (176) such that translation of knife coupling body (174) relative to proximal portion (144) of shaft assembly (140) translates knife member (176) relative to shaft assembly (140).

As best seen in FIGS. 4B-4C and 5B-5C, knife coupling body (174) is coupled to a knife actuation assembly (168) such that as knife trigger (128) pivots toward body (122) and/or pistol grip (124), knife actuation assembly (168) drives knife coupling body (174) distally, thereby driving knife member (176) distally within knife pathway (192). Because knife coupling body (174) is coupled to knife member (176), knife member (176) translates distally within shaft assembly (140), articulation section (110), and within knife pathway (192) of end effector (180), as best shown between FIGS. 5B-5C. Knife member (176) includes distal cutting edge (178) that is configured to sever tissue captured between jaws (182, 184). Therefore, pivoting knife trigger (128) causes knife member (176) to actuate within knife pathway (192) of end effector (180) to sever tissue captured between jaws (182, 184).

Knife trigger (128) is biased to the positions seen in FIGS. 4A-4B (associated with the knife member (176) in the retracted position) by a bias arm (129). Bias arm (129) may include any suitable biasing mechanism as would be apparent to one having ordinary skill in the art in view of the teachings herein. For instance, bias arm (129) may include a torsion spring. Therefore, if an operator releases knife trigger (128), bias arm (129) returns knife trigger (128) to the position shown in FIGS. 4A-4B, thereby translating knife member (176) toward the retracted position.

With distal cutting edge (178) of knife member (176) actuated to the advance position (position shown in FIG. 5C), an operator may press activation button (130) to selectively activate electrode surfaces (194, 196) of jaws (182, 184) to weld/seal severed tissue that is captured between jaws (182, 184). It should be understood that the operator may also press activation button (130) to selectively activate electrode surfaces (194, 196) of jaws (182, 184) at any suitable time during exemplary use. Therefore, the operator may also press activation button (130) while knife member (176) is retracted as shown in FIGS. 3A-3B. Next, the operator may release jaw closure trigger (128) such that jaws (182, 184) pivot into the opened configuration, releasing tissue.

### II. Example of Optical Sensor Electronics for Electrosurgical System

As mentioned above, end effector (180) is configured to grasp, sever, and weld/seal tissue. In particular, jaw (184) may pivot relative to jaw (182) in order to grasp tissue, while knife member (176) is configured to actuate within jaws (182, 184) in order to sever tissue that is grasped between jaws (182, 184). Electrode surfaces (194, 196) may be activated while jaws (182, 184) grasp tissue in order to weld/seal tissue captured between jaws (182, 184). In some instances, it may be desirable to equip end effector (180) with one or more optical sensors for detecting tissue before, during, and/or after grasping, severing, and/or welding/sealing the tissue. In such cases, various electronic components may be incorporated into and/or associated with a surgical instrument, such as a surgical instrument (100), to support the functionality of the optical sensors, such as a light source and a light reader, for example. It may be desirable to consolidate the optical sensor supporting electronic components such as optoelectronics with each other and/or to integrate the optical sensor supporting electronic components with one or more other supporting electronic components of electrosurgical instrument (100). Each of the optical detection-enabling pass-through devices described below provides one or more of these functionalities.

### A. Exemplary Electrosurgical System with Pass-Through Box Containing Optical Sensor Electronics

FIGS. 6-8 show an exemplary electrosurgical system (300) including a second exemplary electrosurgical instrument (302) operatively coupled to a power source in the form of an RF generator (304) via a cable (306) and an optical detection-enabling pass-through device in the form of a pass-through box (308). Electrosurgical instrument (302) is similar to electrosurgical instrument (100) described above except as otherwise described below. In this regard, electrosurgical instrument (302) of this example includes a handle assembly (310), a shaft assembly (312), an articulation assembly (not shown), and an end effector (316) that is operable to grasp, cut, and seal or weld tissue (e.g., a blood vessel, etc.) by applying bipolar RF energy provided by generator (304) to tissue via electrodes (not shown). Electrosurgical instrument (302) may include one or more optical sensors (not shown) positioned on end effector (316) for detecting tissue. Such optical sensors may be configured in accordance with any one or more teachings of U.S. Pat. App. No. [Atty. Ref. END9392USNP1], entitled “Electrosurgical Instrument with Light Accumulator End Effector and Fiber Optics,” filed on even date herewith, the disclosure of which is incorporated by reference herein.

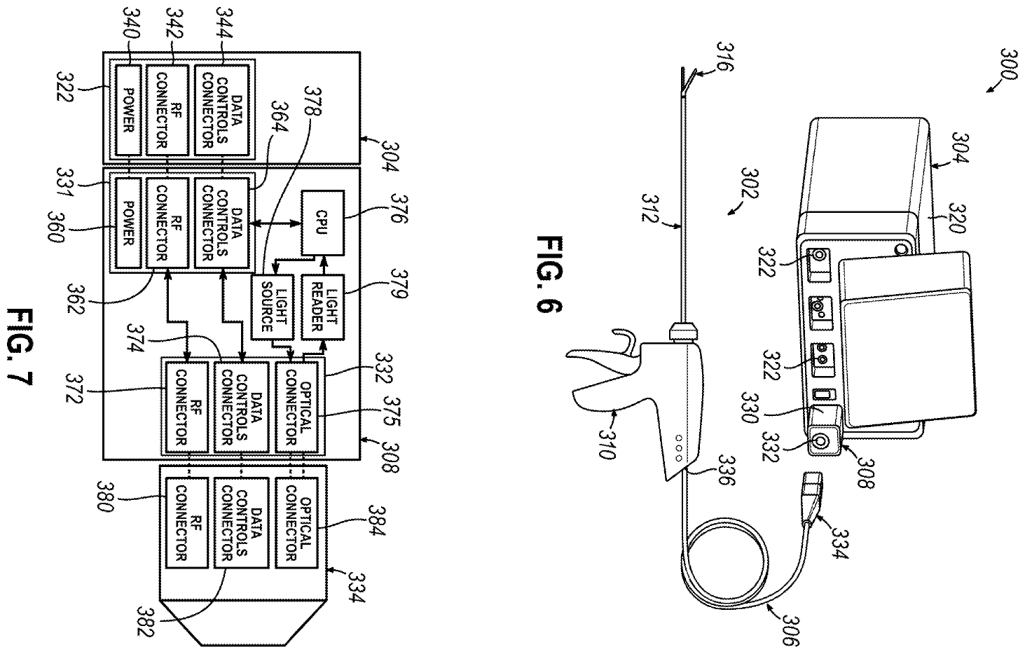

As best shown in FIG. 6, generator (304) of this example includes a console (320) having a plurality of coupling members in the form of generator ports (322) for selectively coupling generator (304) to one or more cables, such as cable (306), and/or to one or more pass-through boxes, such as pass-through box (308). In this regard, pass-through box (308) of the present version includes a pass-through box housing (330) and a proximal coupling member in the form of a pass-through box plug (331) (FIG. 7) configured to be removably received within one of generator ports (322) to thereby selectively couple pass-through box (308) to generator (304). Pass-through box (308) also includes distal coupling member in the form of a pass-through box port (332) for selectively coupling pass-through box (308) to cable (306). To that end, cable (306) of this example includes a proximal coupling member in the form of a proximal cable plug (334) configured to be removably received within pass-through box port (332) to thereby selectively couple cable (306) to pass-through box (308). Cable (306) also includes a distal end (336) fixedly secured to electrosurgical instrument (302) for providing electrosurgical instrument (302) with RF energy delivery and optical detection capabilities. In other versions, cable (306) may be removably coupled to electrosurgical instrument (302).

As best shown in FIG. 7, at least one generator port (322) of generator (304) includes a power connector (340) configured to supply power to pass-through box (308). Generator port (322) also includes at least one RF connector(s) (342) configured to transmit RF energy to and/or from pass-through box (308), and at least one data/controls connector(s) (344) configured to transmit data/control signals to and/or from pass-through box (308).

Pass-through box plug (331) of pass-through box (308) includes a power connector (360) configured to operatively engage power connector (340) of generator (304) to receive power therefrom. Pass-through box plug (331) also includes at least one proximal RF connector(s) (362) configured to operatively engage RF connector(s) (342) of generator (304) to transmit RF energy therebetween, and at least one proximal data/controls connector(s) (364) configured to operatively engage data/controls connector(s) (344) of generator (304) to transmit data/control signals therebetween. Pass-through box port (332) of pass-through box (308) includes at least one distal RF connector(s) (372) operatively coupled to proximal RF connector(s) (362) to transmit RF energy therebetween and configured to transmit such RF energy to and/or from cable (306). Pass-through box port (332) also includes at least one distal data/controls connector(s) (374) operatively coupled to proximal data/controls connector(s) (364) to transmit data/control signals therebetween and configured to transmit such data/control signals to and/or from cable (306). In the example shown, pass-through box port (332) further includes at least one optical connector(s) (375), such as at least one optical fiber connector(s).

In this regard, pass-through box (308) of the present version also includes various optical sensor supporting electronic components, including a processor (376) operatively coupled to proximal data/controls connector(s) (364) to transmit data/control signals therebetween, a light source (378) operatively coupled to processor (376) to receive control signals therefrom and configured to emit light, and a light reader (379) operatively coupled to processor (376) to send data signals thereto and configured to generate light data based on light received by light reader (379). As shown, optical connector(s) (375) is operatively coupled to light source (378) (e.g., via optical fiber(s)) to receive light emitted therefrom, and is configured to transmit light received from light source (378) to cable (306). Optical connector(s) (375) is also configured to receive light from cable (306), and is operatively coupled to light reader (379) (e.g., via optical fiber(s)) to transmit light received from cable (306) to light reader (379).

Proximal cable plug (334) of cable (306) includes at least one RF connector(s) (380) configured to operatively engage distal RF connector(s) (372) of pass-through box (308) to transmit RF energy therebetween. Proximal cable plug (334) also includes at least one data/controls connector(s) (382) configured to operatively engage distal data/controls connector(s) (374) of pass-through box (308) to transmit data/control signals therebetween, and at least one optical connector(s) (384)), such as at least one optical fiber connector(s), operatively coupled to the optical sensors of end effector (316) (e.g., via optical fiber(s)) and configured to operatively engage optical connector(s) (375) of pass-through box (308) to transmit light therebetween.

Thus, pass-through box (308) may cooperate with generator (304) to enable electrosurgical instrument (302) to perform both RF energy delivery and optical detection. More particularly, RF energy delivery may be achieved by transmitting RF energy between generator (304) and the electrodes of end effector (316) via RF connectors (342, 362, 372, 380). Optical detection may be achieved by emitting light from light source (378), directing the light distally from light source (378) to the optical sensors of end effector (316) via optical connectors (375, 384)), and subsequently directing the light proximally from the optical sensors of end effector (316) to light reader (379) via optical connectors (375, 384)). Light reader (379) may generate light data based on the light received thereby and transmit such light data to processor (376), which may be configured to interpret the light data in order to determine a status of tissue, such as a property of the tissue and/or a position of the tissue relative to end effector (316), for example. In this manner, light emission, light reading, and light data interpretation may be performed directly within pass-through box (308). Processor (376) may, in turn, transmit the determined tissue status to console (320) of generator (304) via data/controls connectors (344, 364) for communicating the determined status to the operator and/or for further processing (e.g., to automatically take a predetermined action in response to the determined tissue status, such as initiating, adjusting, or terminating RF energy delivery). In the example shown, processor (376), light source (378), and light reader (379) are each powered by generator (304) via power connectors (340, 360). In other versions, pass-through box (308) may include one or more power sources, such as batteries (not shown), for powering any one or more of processor (376), light source (378), and/or light reader (379).

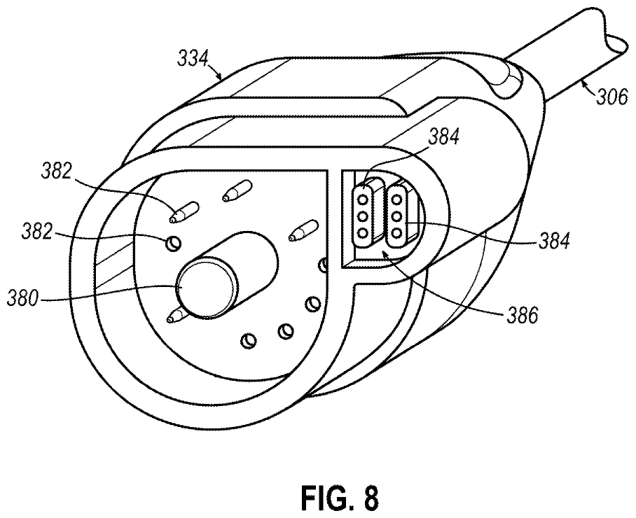

As best shown in FIG. 8, optical connectors (384) of proximal cable plug (334) are arranged relative to RF connector(s) (380) and data/controls connectors (382) to define a key portion (386) of proximal cable plug (334). In this regard, key portion (386) may be sized and configured to mate with a corresponding keyway portion (not shown) of pass-through box port (332) to thereby permit insertion of proximal cable plug (334) into pass-through box port (332) and to inhibit insertion of proximal cable plug (334) into any of the generator ports (322). Thus, key portion (386) may prevent cable (306) from being directly coupled to generator (304) without pass-through box (308) positioned therebetween, to ensure that the optical sensors of end effector (316) are properly supported by the optical sensor supporting electronic components of pass-through box (308).

While the optical detection-enabling pass-through device of this example has been described in the form of pass-through box (308), it will be appreciated that the pass-through device may have any other suitable form, such as a cable or a portion of a cable, for example. Also, while various coupling members have been described in the form of ports (322, 332) and corresponding plugs (331, 334), it will be appreciated that any other suitable types of coupling members may be used. In some versions, the aforementioned ports (322, 332) may each be replaced with plugs, and the aforementioned plugs (331, 334) may each be replaced with ports. In addition, or alternatively, RF connectors (342, 362, 372, 380) or other suitable connectors may be used to supply energy to a harmonic transducer (not shown) of electrosurgical instrument (302), such as to seal or weld tissue via an ultrasonic blade (not shown).

It will be appreciated that by containing the optical sensor supporting electronic components, pass-through box (308) may provide a reduction in size and/or cost of electrosurgical instrument (302), at least by comparison to an electrosurgical instrument containing such optical sensor supporting electronic components. Moreover, pass-through box (308) may be reusable and may be compatible with multiple electrosurgical instruments (302) (e.g., having either the same or a different configuration from that shown) for enabling optical detection. In some instances, pass-through box (308) may be readily replaced by another pass-through box having upgraded electronics, for example, without requiring replacement of electrosurgical instrument (302). It will also be appreciated that pass-through box (308) may be positioned outside of the sterile field during a surgical operation, such that pass-through box (308) may not be subjected to sterilization processes.

### B. Exemplary Electrosurgical System with Pass-Through Box for Splitting Transmission of Light and RF Energy

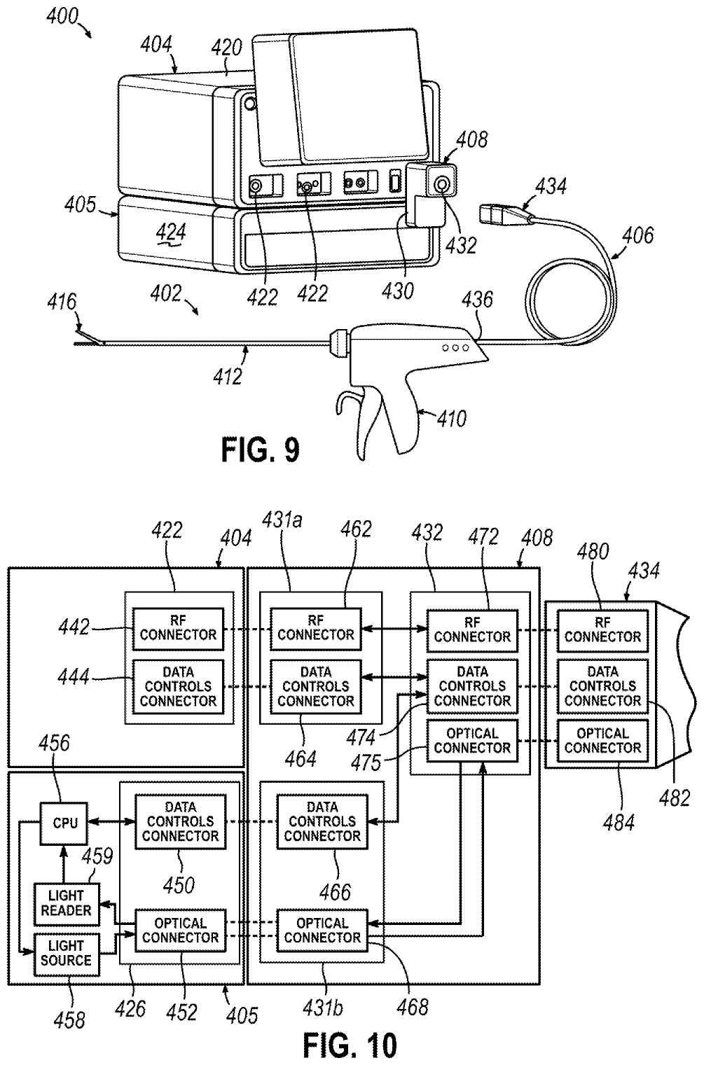

FIGS. 9-10 show another exemplary electrosurgical system (400) including a third exemplary electrosurgical instrument (402) operatively coupled to a power source in the form of an RF generator (404) and to a spectrometer (405) via a cable (406) and an optical detection-enabling pass-through device in the form of a pass-through box (408). Electrosurgical instrument (402) is similar to electrosurgical instrument (100) described above except as otherwise described below. In this regard, electrosurgical instrument (402) of this example includes a handle assembly (410), a shaft assembly (412), an articulation assembly (not shown), and an end effector (416) that is operable to grasp, cut, and seal or weld tissue (e.g., a blood vessel, etc.) by applying bipolar RF energy provided by generator (404) to tissue via electrodes (not shown). Electrosurgical instrument (402) may include one or more optical sensors (not shown) positioned on end effector (416) for detecting tissue. Such optical sensors may be configured in accordance with any one or more teachings of U.S. Pat. App. No. [Atty. Ref. END9392USNP1], entitled “Electrosurgical Instrument with Light Accumulator End Effector and Fiber Optics,” filed on even date herewith, the disclosure of which is incorporated by reference herein.

As best shown in FIG. 9, generator (404) of this example includes a console (420) having a plurality of coupling members in the form of generator ports (422) for selectively coupling generator (404) to one or more cables, such as cable (406), and/or to one or more pass-through boxes, such as pass-through box (408). Spectrometer (405) includes a housing (424) having a coupling member in the form of a spectrometer port (426) (FIG. 10) for selectively coupling spectrometer (405) to a cable, such as cable (406), and/or to a pass-through box, such as pass-through box (408). In this regard, pass-through box (408) of the present version includes a pass-through box housing (430) and an upper, proximal coupling member in the form of an upper, proximal pass-through box plug (431a) (FIG. 10) configured to be removably received within one of generator ports (422) to thereby selectively couple pass-through box (408) to generator (404). Pass-through box (408) further includes a lower, proximal coupling member in the form of a lower, proximal pass-through box plug (431b) (FIG. 10) configured to be removably received within spectrometer port (426) to thereby selectively couple pass-through box (408) to spectrometer (405). Upper and lower pass-through box plugs (431a, 431b) may be positioned relative to each other to enable simultaneous coupling of pass-through box (408) to generator (404) and spectrometer (405), at least when generator (404) is positioned on top of spectrometer (405) as shown. Pass-through box (408) also includes a distal coupling member in the form of a pass-through box port (432) for selectively coupling pass-through box (408) to cable (406). To that end, cable (406) of this example includes a proximal coupling member in the form of a proximal cable plug (434) configured to be removably received within pass-through box port (432) to thereby selectively couple cable (406) to pass-through box (408). Cable (406) also includes a distal end (436) fixedly secured to electrosurgical instrument (402) for providing electrosurgical instrument (402) with RF energy delivery and optical detection capabilities. In other versions, cable (406) may be removably coupled to electrosurgical instrument (402).

As best shown in FIG. 10, at least one generator port (422) of generator (404) includes at least one RF connector(s) (442) configured to transmit RF energy to and/or from pass-through box (408). Generator port (422) also includes at least one data/controls connector(s) (444) configured to transmit data/control signals to and/or from pass-through box (408). In some versions, generator port (422) may also include a power connector (not shown) configured to supply power to spectrometer (405), for example.

Spectrometer port (426) of spectrometer (405) includes at least one data/controls connector (450) configured to transmit data/control signals to and/or from pass-through box (408). In the example shown, spectrometer port (426) further includes at least one optical connector(s) (452), such as at least one optical fiber connector(s). In this regard, spectrometer (405) of the present version also includes various optical sensor supporting electronic components, including a processor (456) operatively coupled to data/controls connector(s) (450) to transmit data/control signals therebetween, a light source (458) operatively coupled to processor (456) to receive control signals therefrom and configured to emit light, and a light reader (459) operatively coupled to processor (456) to send data signals thereto and configured to generate light data based on light received by light reader (459). As shown, optical connector(s) (452) is operatively coupled to light source (458) (e.g., via optical fiber(s)) to receive light emitted therefrom, and is configured to transmit light received from light source (458) to pass-through box (408). Optical connector(s) (452) is also configured to receive light from pass-through box (408), and is operatively coupled to light reader (459) (e.g., via optical fiber(s)) to transmit light received from pass-through box (408) to light reader (459).

Upper pass-through box plug (431a) of pass-through box (408) includes at least one proximal RF connector(s) (462) configured to operatively engage RF connector(s) (442) of generator (404) to transmit RF energy therebetween. Upper pass-through box plug (431a) also includes at least one upper, proximal data/controls connector(s) (464) configured to operatively engage data/controls connector(s) (444) of generator (404) to transmit data/control signals therebetween. Lower pass-through box plug (431b) of pass-through box (408) includes at least one lower, proximal data/controls connector(s) (466) configured to operatively engage data/controls connector(s) (450) of spectrometer (405) to transmit data/control signals therebetween. Lower pass-through box plug (431b) also includes at least one proximal optical connector(s) (468), such as at least one optical fiber connector(s), configured to operatively engage optical connector(s) (452) of spectrometer (405) to transmit light therebetween.

Pass-through box port (432) of pass-through box (408) includes at least one distal

RF connector(s) (472) operatively coupled to proximal RF connector(s) (462) to transmit RF energy therebetween and configured to transmit such RF energy to and/or from cable (406). Pass-through box port (432) also includes at least one distal data/controls connector(s) (474) operatively coupled to upper and lower proximal data/controls connectors (464, 466) to transmit data/control signals therebetween and configured to transmit such data/control signals to and/or from cable (406). Pass-through box port (432) further includes at least one distal optical connector(s) (475), such as at least one optical fiber connector(s), operatively coupled to proximal optical connector(s) (468) (e.g., via optical fiber(s)) to transmit light therebetween and configured to transmit light to and from cable (406).

Proximal cable plug (434) of cable (406) includes at least one RF connector(s) (480) configured to operatively engage distal RF connector(s) (472) of pass-through box (408) to transmit RF energy therebetween. Proximal cable plug (434) also includes at least one data/controls connector(s) (482) configured to operatively engage distal data/controls connector(s) (474) of pass-through box (408) to transmit data/control signals therebetween, and at least one optical connector(s) (484), such as at least one optical fiber connector(s), operatively coupled to the optical sensors of end effector (416) (e.g., via optical fiber(s)) and configured to operatively engage distal optical connector(s) (475) of pass-through box (408) to transmit light therebetween.

Thus, pass-through box (408) may cooperate with both generator (404) and spectrometer (405) to enable electrosurgical instrument (402) to perform both RF energy delivery and optical detection. More particularly, RF energy delivery may be achieved by transmitting RF energy between generator (404) and the electrodes of end effector (416) via RF connectors (442, 462, 472, 480). Optical detection may be achieved by emitting light from light source (458), directing the light distally from light source (458) to the optical sensors of end effector (416) via optical connectors (452, 468, 475, 484), and subsequently directing the light proximally from the optical sensors of end effector (416) to light reader (459) via optical connectors (452, 468, 475, 484). Light reader (459) may generate light data based on the light received thereby and transmit such light data to processor (456), which may be configured to interpret the light data in order to determine a status of tissue, such as a property of the tissue and/or a position of the tissue relative to end effector (416), for example. In this manner, light emission, light reading, and light data interpretation may be performed within spectrometer (405), rather than directly within pass-through box (408). Processor (456) may, in turn, transmit the determined tissue status to console (420) of generator (404) via data/controls connectors (444, 450, 464, 466) for communicating the determined status to the operator and/or for further processing (e.g., to automatically take a predetermined action in response to the determined tissue status, such as initiating, adjusting, or terminating RF energy delivery). In some versions, processor (456), light source (458), and light reader (459) may each be powered by one or more power sources, such as batteries (not shown), of spectrometer (405). In other versions, any one or more of processor (456), light source (458), and/or light reader (459) may be powered by generator (404) via respective power connectors (not shown).

While the optical detection-enabling pass-through device of this example has been described in the form of pass-through box (408), it will be appreciated that the pass-through device may have any other suitable form, such as a cable or a portion of a cable, for example. Also, while various coupling members have been described in the form of ports (422, 426, 432) and corresponding plugs (431a, 43b, 434), it will be appreciated that any other suitable types of coupling members may be used. In some versions, the aforementioned ports (422, 426, 432) may each be replaced with plugs, and the aforementioned plugs (431a, 431b, 434) may each be replaced with ports. In addition, or alternatively, RF connectors (442, 462, 472, 480) or other suitable connectors may be used to supply energy to a harmonic transducer (not shown) of electrosurgical instrument (402), such as to seal or weld tissue via an ultrasonic blade (not shown).

It will be appreciated that by containing the optical sensor supporting electronic components, pass-through box (408) may provide a reduction in size and/or cost of electrosurgical instrument (402), at least by comparison to an electrosurgical instrument containing such optical sensor supporting electronic components. Moreover, pass-through box (408) may be reusable and may be compatible with multiple electrosurgical instruments (402) (e.g., having either the same or a different configuration from that shown) for enabling optical detection. In some instances, pass-through box (408) may be readily replaced by another pass-through box having upgraded electronics, for example, without requiring replacement of electrosurgical instrument (402). It will also be appreciated that pass-through box (408) may be positioned outside of the sterile field during a surgical operation, such that pass-through box (408) may not be subjected to sterilization processes.

### C. Exemplary Electrosurgical System with Cable Plug Containing Optical Sensor Electronics

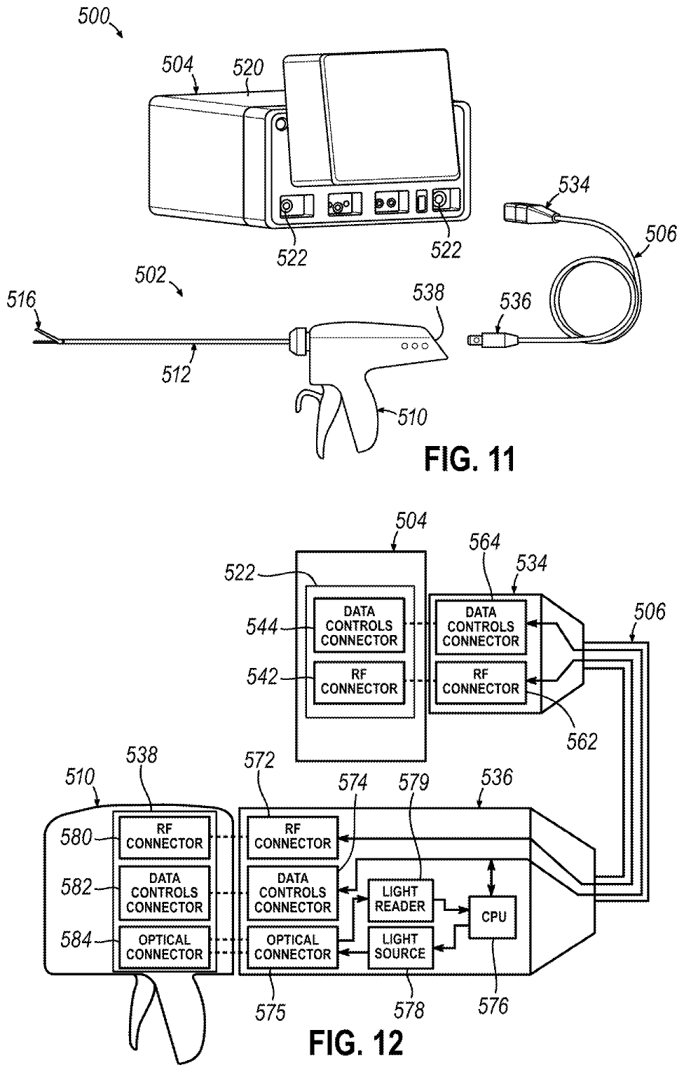

FIGS. 11-12 show another exemplary electrosurgical system (500) including a fourth exemplary electrosurgical instrument (502) operatively coupled to a power source in the form of an RF generator (504) via a cable (506). Electrosurgical instrument (502) is similar to electrosurgical instrument (100) described above except as otherwise described below. In this regard, electrosurgical instrument (502) of this example includes a handle assembly (510), a shaft assembly (512), an articulation assembly (not shown), and an end effector (516) that is operable to grasp, cut, and seal or weld tissue (e.g., a blood vessel, etc.) by applying bipolar RF energy provided by generator (504) to tissue via electrodes (not shown). Electrosurgical instrument (502) may include one or more optical sensors (not shown) positioned on end effector (516) for detecting tissue. Such optical sensors may be configured in accordance with any one or more teachings of U.S. Pat. App. No. [Atty. Ref. END9392USNP1], entitled “Electrosurgical Instrument with Light Accumulator End Effector and Fiber Optics,” filed on even date herewith, the disclosure of which is incorporated by reference herein.

As best shown in FIG. 11, generator (504) of this example includes a console (520) having a plurality of coupling members in the form of generator ports (522) for selectively coupling generator (504) to one or more cables, such as cable (506). In this regard, cable (506) of the present version includes a proximal coupling member in the form of a proximal cable plug (534) configured to be removably received within one of generator ports (522) to thereby selectively couple cable (506) to generator (504). Cable (506) also includes an integrated distal coupling member and optical detection-enabling pass-through device in the form of a distal cable plug (536) for selectively coupling cable (506) to electrosurgical instrument (502). To that end, handle assembly (510) of electrosurgical instrument (502) includes a coupling member in the form of an instrument port (538) configured to removably receive distal cable plug (536) to thereby selectively couple electrosurgical instrument (502) to cable (506) for providing electrosurgical instrument (502) with RF energy delivery and optical detection capabilities.

As best shown in FIG. 12, at least one generator port (522) of generator (504) includes at least one RF connector(s) (542) configured to transmit RF energy to and/or from cable (506). Generator port (522) also includes at least one data/controls connector(s) (544) configured to transmit data/control signals to and/or from cable (506). In some versions, generator port (522) may also include a power connector (not shown) configured to supply power to cable (506), for example.

Proximal cable plug (534) of cable (506) includes at least one proximal RF connector(s) (562) configured to operatively engage RF connector(s) (542) of generator (504) to transmit RF energy therebetween, and at least one proximal data/controls connector(s) (564) configured to operatively engage data/controls connector(s) (544) of generator (504) to transmit data/control signals therebetween. Distal cable plug (536) of cable (506) includes at least one distal RF connector(s) (572) operatively coupled to proximal RF connector(s) (562) to transmit RF energy therebetween and configured to transmit such RF energy to and/or from electrosurgical instrument (502). Distal cable plug (536) also includes at least one distal data/controls connector(s) (574) operatively coupled to proximal data/controls connector(s) (564) to transmit data/control signals therebetween and configured to transmit such data/control signals to and/or from electrosurgical instrument (502). In the example shown, distal cable plug (536) further includes at least one optical connector(s) (575), such as at least one optical fiber connector(s).

In this regard, distal cable plug (536) of the present version also includes various optical sensor supporting electronic components, including a processor (576) operatively coupled to proximal and distal data/controls connector(s) (564, 574) to transmit data/control signals therebetween, a light source (578) operatively coupled to processor (576) to receive control signals therefrom and configured to emit light, and a light reader (579) operatively coupled to processor (576) to send data signals thereto and configured to generate light data based on light received by light reader (579). As shown, optical connector(s) (575) is operatively coupled to light source (578) (e.g., via optical fiber(s)) to receive light emitted therefrom, and is configured to transmit light received from light source (578) to electrosurgical instrument (502). Optical connector(s) (575) is also configured to receive light from electrosurgical instrument (502), and is operatively coupled to light reader (579) (e.g., via optical fiber(s)) to transmit light received from electrosurgical instrument (502) to light reader (579).

Instrument port (538) of electrosurgical instrument (502) includes at least one RF connector(s) (580) configured to operatively engage distal RF connector(s) (572) of distal cable plug (536) to transmit RF energy therebetween. Instrument port (538) also includes at least one data/controls connector(s) (582) configured to operatively engage distal data/controls connector(s) (574) of distal cable plug (536) to transmit data/control signals therebetween, and at least one optical connector(s) (584), such as at least one optical fiber connector(s), operatively coupled to the optical sensors of end effector (516) (e.g., via optical fiber(s)) and configured to operatively engage optical connector(s) (575) of distal cable plug (536) to transmit light therebetween.

Thus, distal cable plug (536) may cooperate with generator (504) to enable electrosurgical instrument (502) to perform both RF energy delivery and optical detection. More particularly, RF energy delivery may be achieved by transmitting RF energy between generator (504) and the electrodes of end effector (516) via RF connectors (542, 562, 572, 580). Optical detection may be achieved by emitting light from light source (578), directing the light distally from light source (578) to the optical sensors of end effector (516) via optical connectors (575, 584), and subsequently directing the light proximally from the optical sensors of end effector (516) to light reader (579) via optical connectors (575, 584). Light reader (579) may generate light data based on the light received thereby and transmit such light data to processor (576), which may be configured to interpret the light data in order to determine a status of tissue, such as a property of the tissue and/or a position of the tissue relative to end effector (516), for example. In this manner, light emission, light reading, and light data interpretation may be performed directly within distal cable plug (536). Processor (576) may, in turn, transmit the determined tissue status to console (520) of generator (504) via data/controls connectors (544, 564) for communicating the determined status to the operator and/or for further processing (e.g., to automatically take a predetermined action in response to the determined tissue status, such as initiating, adjusting, or terminating RF energy delivery). In some versions, processor (576), light source (578), and light reader (579) may each be powered by one or more power sources, such as batteries (not shown), of distal cable plug (536). In other versions, any one or more of processor (576), light source (578), and/or light reader (579) may be powered by generator (504) via respective power connectors (not shown).

While the optical detection-enabling pass-through device of this example has been described in the form of distal cable plug (536), it will be appreciated that the pass-through device may have any other suitable form. For example, the pass-through device may be integrated with any other portion of cable (506), such as proximal cable plug (534). Also, while various coupling members have been described in the form of ports (522, 538) and corresponding plugs (534, 536), it will be appreciated that any other suitable types of coupling members may be used. In some versions, the aforementioned ports (522, 538) may each be replaced with plugs, and the aforementioned plugs (534, 536) may each be replaced with ports. In addition, or alternatively, RF connectors (542, 562, 572, 580) or other suitable connectors may be used to supply energy to a harmonic transducer (not shown) of electrosurgical instrument (502), such as to seal or weld tissue via an ultrasonic blade (not shown).

It will be appreciated that by containing the optical sensor supporting electronic components, distal cable plug (536) may provide a reduction in size and/or cost of electrosurgical instrument (502), at least by comparison to an electrosurgical instrument containing such optical sensor supporting electronic components. Moreover, cable (506) may be reusable and may be compatible with multiple electrosurgical instruments (502) (e.g., having either the same or a different configuration from that shown) for enabling optical detection. In some instances, cable (506) may be readily replaced by another cable having upgraded electronics, for example, without requiring replacement of electrosurgical instrument (502). It will also be appreciated that distal cable plug (536) may allow the length of optical fiber(s) to be minimized due to the relatively close proximity of distal cable plug (536) to end effector (516) and may thereby provide improved light transmission.

### D. Exemplary Electrosurgical System with Removable Instrument Body Containing Optical Sensor Electronics

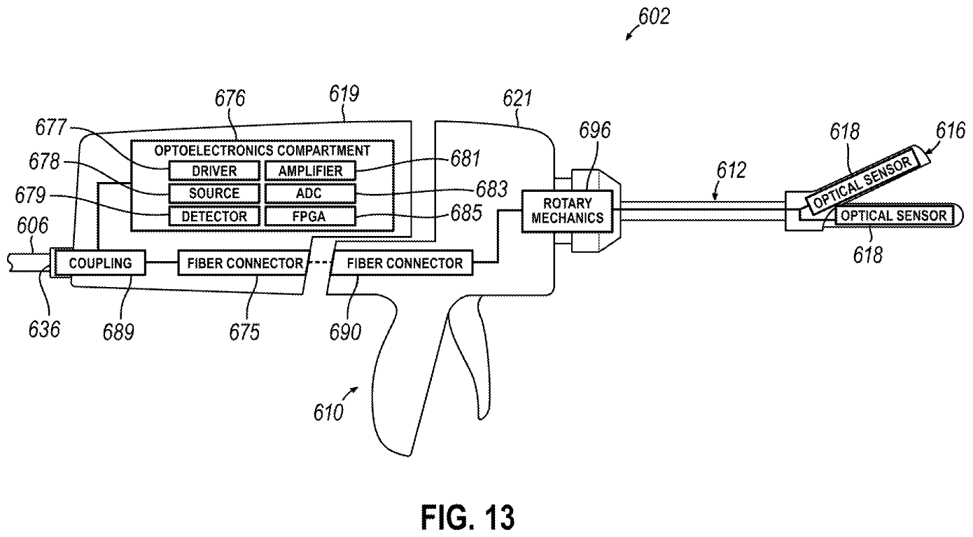

FIG. 13 shows a fifth exemplary electrosurgical instrument (602) configured to be operatively coupled to a power source (not shown) via a cable (606). Electrosurgical instrument (602) is similar to electrosurgical instrument (100) described above except as otherwise described below. In this regard, electrosurgical instrument (602) of this example includes a handle assembly (610), a shaft assembly (612), an articulation assembly (not shown), and an end effector (616) that is operable to grasp, cut, and seal or weld tissue (e.g., a blood vessel, etc.) by applying bipolar RF energy provided by generator (604) to tissue via electrodes (not shown). Electrosurgical instrument (602) includes a plurality of optical sensors (618) positioned on end effector (616) for detecting tissue. Optical sensors (618) may each include collimation optics and a lightbox (not shown), for example. Optical sensors (618) may be configured in accordance with any one or more teachings of U.S. Pat. App. No. [Atty. Ref. END9392USNP1], entitled “Electrosurgical Instrument with Light Accumulator End Effector and Fiber Optics,” filed on even date herewith, the disclosure of which is incorporated by reference herein.

As shown, handle assembly (610) of this example includes an optical detection-enabling pass-through device in the form of a proximal body (619) removably coupled to a distal body (621), each configured to house or otherwise support various components of electrosurgical instrument (602) in manners similar to that described above in connection with FIGS. 1-5C. In the example shown, cable (606) includes a distal end (636) fixedly secured to proximal body (619) for providing electrosurgical instrument (302) with RF energy delivery capabilities. In other versions, cable (606) may be removably coupled to proximal body (619).

Proximal body (619) of handle assembly (610) includes at least one optical fiber connector(s) (675). In this regard, proximal body (619) of the present version also includes an optoelectronics compartment (676) housing various optical sensor supporting electronic components, including a light source driver (677), a light source (678) configured to emit light, a light detector (679) configured to generate light data based on light received by light detector (679), an optical amplifier (681), an analog-to-digital converter (“ADC”) (683), and a field-programmable gate array (“FPGA”) (685). In some versions, a processor (not shown) may be operatively coupled to light source driver (677) to send control signals thereto, and may be operatively coupled to light detector (679) to receive data signals therefrom. Optical fiber connector(s) (675) is operatively coupled to optoelectronics compartment (676) via at least one optical fiber(s) (not shown) and a coupling (689) to receive light emitted from light source (678), and is configured to transmit light received from light source (678) to distal body (621). Optical fiber connector(s) (675) is also configured to receive light from distal body (621), and is operatively coupled to optoelectronics compartment (676) via the at least one optical fiber(s) and coupling (689) to transmit light received from distal body (621) to light detector (679). Coupling (689) may include any suitable coupling mechanics and/or coupling optics to facilitate such transmission of light.

Distal body (621) of handle assembly (610) includes at least one optical fiber connector(s) (690) operatively coupled to optical sensors (618) of end effector (616) via one or more optical fiber(s) (not shown) and a fiber optic rotary coupling (696), and configured to operatively engage optical fiber connector(s) (675) of proximal body (619) to transmit light therebetween. Fiber optic rotary coupling (696) may be configured in accordance with any one or more teachings of U.S. Pat. App. No. [Atty. Ref. END9390USNP1], entitled “Electrosurgical Instrument with Fiber Optic Rotary Coupling,” filed on even date herewith, the disclosure of which is incorporated by reference herein.

Thus, proximal body (619) may cooperate with a generator to enable electrosurgical instrument (602) to perform both RF energy delivery and optical detection. More particularly, RF energy delivery may be achieved by transmitting RF energy between the generator and the electrodes of end effector (616) via respective RF connectors (not shown). Optical detection may be achieved by emitting light from light source (678), directing the light distally from light source (678) to optical sensors (618) of end effector (616) via optical fiber connectors (675, 690), and subsequently directing the light proximally from optical sensors (618) of end effector (616) to light detector (679) via optical connectors (675, 690). Light detector (679) may generate light data based on the light received thereby and transmit such light data to the processor, which may be configured to interpret the light data in order to determine a status of tissue, such as a property of the tissue and/or a position of the tissue relative to end effector (616), for example. In this manner, light emission, light reading, and light data interpretation may be performed directly within proximal body (619). The processor may, in turn, transmit the determined tissue status to a console of the generator via respective data/controls connectors (not shown) for communicating the determined status to the operator and/or for further processing (e.g., to automatically take a predetermined action in response to the determined tissue status, such as initiating, adjusting, or terminating RF energy delivery). In some versions, the processor, light source (678), and light detector (679) may each be powered by one or more power sources, such as batteries (not shown), of proximal body (619). In other versions, any one or more of the processor, light source (678), and/or light reader (679) may be powered by the generator via respective power connectors (not shown).

While the optical detection-enabling pass-through device of this example has been described in the form of proximal body (619), it will be appreciated that the pass-through device may have any other suitable form. For example, the pass-through device may be integrated with any other portion of handle assembly (610), such as proximal distal body (621). In addition, or alternatively, the RF connectors or other suitable connectors may be used to supply energy to a harmonic transducer (not shown) of electrosurgical instrument (602), such as to seal or weld tissue via an ultrasonic blade (not shown).

### III. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A surgical system, comprising: (a) a surgical instrument, comprising: (i) a shaft assembly having a distal end, and (ii) an end effector at the distal end of the shaft assembly, the end effector including: (A) a first jaw, (B) a second jaw movably coupled relative to the first jaw for clamping tissue therebetween, and (C) at least one optical sensor for detecting the tissue; (b) a generator configured to supply a therapeutic energy to at least one of the first jaw or the second jaw; and (c) a pass-through device configured to be connected between the surgical instrument and the generator, the pass-through device comprising: (i) at least one therapeutic energy connector configured to operatively couple the generator to the surgical instrument for transmitting the therapeutic energy from the generator to the at least one of the first jaw or the second jaw, and (ii) at least one optical component configured to transmit light to the at least one optical sensor and to receive light from the at least one optical sensor.

### EXAMPLE 2

The surgical system of Example 1, wherein the at least one optical component includes at least one optical fiber connector.

### EXAMPLE 3

The surgical system of any one or more of Examples 1 through 2, wherein the at least one optical component includes at least one optoelectronic component.

### EXAMPLE 4

The surgical system of Example 3, wherein the at least one optoelectronic component includes a light source configured to transmit light to the at least one optical sensor.

### EXAMPLE 5

The surgical system of Example 4, wherein the at least one optoelectronic component further includes a light reader configured to receive light from the at least one optical sensor.

### EXAMPLE 6

The surgical system of any one or more of Examples 3 through 5, wherein the pass-through device further comprises at least one power connector configured to supply power from the generator to the at least one optoelectronic component.

### EXAMPLE 7

The surgical system of any one or more of Examples 3 through 6, wherein the pass-through device further comprises a power source configured to supply power to the at least one optoelectronic component.

### EXAMPLE 8

The surgical system of any one or more of Examples 1 through 7, wherein the pass-through device further comprises at least one data connector configured to operatively couple the generator to the surgical instrument for transmitting data signals therebetween.

### EXAMPLE 9

The surgical system of Example 8, wherein the pass-through device further comprises a processor operatively coupled to the at least one optical component and to the at least one data connector, wherein the processor is configured to transmit control signals to the generator for adjusting the therapeutic energy based on data signals received by the processor from the at least one optical component.

### EXAMPLE 10

The surgical system of any one or more of Examples 1 through 9, further comprising a spectrometer, wherein the at least one optical component is configured to operatively couple the spectrometer to the surgical instrument for transmitting light between the spectrometer and the at least one optical sensor.

### EXAMPLE 11

The surgical system of Example 10, wherein the spectrometer includes a light source configured to transmit light to the at least one optical sensor and a light reader configured to receive light from the at least one optical sensor.

### EXAMPLE 12

The surgical system of any one or more of Examples 1 through 11, wherein the pass-through device includes a pass-through box configured to be removably coupled to the surgical instrument and to the generator.

### EXAMPLE 13

The surgical system of any one or more of Examples 1 through 11, further comprising a cable removably coupled to the surgical instrument and to the generator, wherein the pass-through device is presented by the cable.

### EXAMPLE 14

The surgical system of Example 13, wherein the cable includes a distal cable plug removably coupled to the surgical instrument, wherein the pass-through device is presented by the distal cable plug.

### EXAMPLE 15

The surgical system of any one or more of Examples 1 through 11, wherein the surgical instrument includes a proximal body and a distal body removably coupled to each other, wherein the shaft assembly extends distally from the distal body, wherein the pass-through device is presented by the proximal body.

### EXAMPLE 16

A pass-through device for a surgical system, comprising: (a) a housing; (b) a first coupling member affixed to the housing, the first coupling member including at least one first therapeutic energy connector configured to receive therapeutic energy from a generator; and (c) a second coupling member affixed to the housing, the second coupling member including: (i) at least one second therapeutic energy connector operatively coupled to the at least one first therapeutic energy connector to receive the therapeutic energy from the at least one first therapeutic energy connector, and configured to transmit the therapeutic energy to an end effector of a surgical instrument, and (ii) at least one optical fiber connector configured to transmit light to at least one optical sensor of the end effector and to receive light from the at least one optical sensor.

### EXAMPLE 17

The pass-through device of Example 16, further comprising at least one optoelectronic component operatively coupled to the at least one optical fiber connector.

### EXAMPLE 18

The pass-through device of Example 17, wherein the at least one optoelectronic component includes: (i) a light source configured to transmit light to the at least one optical sensor, and (ii) a light reader configured to receive light from the at least one optical sensor.

### EXAMPLE 19

The pass-through device of Example 18, further comprising a processor operatively coupled to the light source and to the light reader, wherein the processor is configured to receive data signals from the light reader, wherein the processor is configured to send control signals to the light source.

### EXAMPLE 20

A cable for a surgical system, comprising: (a) a proximal cable plug; and (b) a distal cable plug, wherein the distal cable plug includes: (i) at least one therapeutic energy connector configured to transmit therapeutic energy between a generator and an end effector of a surgical instrument, (ii) a light source configured to transmit light to at least one optical sensor of the end effector, and (iii) a light reader configured to receive light from the at least one optical sensor.

### EXAMPLE 21

A method, comprising: (a) transmitting a therapeutic energy from a generator to an end effector of a surgical instrument via a pass-through device; (b) transmitting light to an optical sensor of the end effector via the pass-through device; and (c) receiving light from the optical sensor of the end effector via the pass-through device.

### EXAMPLE 22

The method of Example 21, further comprising removably coupling the pass-through device to the generator.

### EXAMPLE 23

The method of any one or more of Examples 21 through 22, further comprising removably coupling the pass-through device to the surgical instrument.

### EXAMPLE 24

The method of any one or more of Examples 21 through 23, wherein the act of transmitting light includes transmitting light from a spectrometer to the optical sensor of the end effector via the pass-through device, wherein the act of receiving light includes transmitting light from the optical sensor of the end effector to the spectrometer via the pass-through device.

### EXAMPLE 25

The method of Example 24, further comprising removably coupling the pass-through device to the spectrometer.

### EXAMPLE 26

The method of any one or more of Examples 21 through 25, further comprising adjusting the therapeutic energy based on the light received from the optical sensor of the end effector via the pass-through device.

### IV. Miscellaneous

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those of ordinary skill in the art.

While the examples herein are described mainly in the context of electrosurgical instruments, it should be understood that various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Additionally, any one or more of the teachings herein may be combined with any one or more of the teachings of U.S. Pat. App. No. [Atty. Ref. END9390USNP1], entitled “Electrosurgical Instrument with Fiber Optic Rotary Coupling,” filed on even date herewith; and U.S. Pat. App. No. [Atty. Ref. END9392USNP1], entitled “Electrosurgical Instrument with Light Accumulator End Effector and Fiber Optics,” filed on even date herewith. The disclosure of each of these US patent documents is incorporated by reference herein.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, Calif. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein, in its entirety.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We Claim:

1. A surgical system, comprising:
(a) a surgical instrument, comprising:
(i) a shaft assembly having a distal end, and
(ii) an end effector at the distal end of the shaft assembly, the end effector including:
(A) a first jaw,
(B) a second jaw movably coupled relative to the first jaw for clamping tissue therebetween, and
(C) at least one optical sensor for detecting the tissue;
(b) a generator configured to supply a therapeutic energy to at least one of the first jaw or the second jaw; and
(c) a pass-through device configured to be connected between the surgical instrument and the generator, the pass-through device comprising:
(i) at least one therapeutic energy connector configured to operatively couple the generator to the surgical instrument for transmitting the therapeutic energy from the generator to the at least one of the first jaw or the second jaw, and p2 (ii) at least one optical component configured to transmit light to the at least one optical sensor and to receive light from the at least one optical sensor.

2. The surgical system of claim 1, wherein the at least one optical component includes at least one optical fiber connector.

3. The surgical system of claim 1, wherein the at least one optical component includes at least one optoelectronic component.

4. The surgical system of claim 3, wherein the at least one optoelectronic component includes a light source configured to transmit light to the at least one optical sensor.

5. The surgical system of claim 4, wherein the at least one optoelectronic component further includes a light reader configured to receive light from the at least one optical sensor.

6. The surgical system of claim 3, wherein the pass-through device further comprises at least one power connector configured to supply power from the generator to the at least one optoelectronic component.

7. The surgical system of claim 3, wherein the pass-through device further comprises a power source configured to supply power to the at least one optoelectronic component.

8. The surgical system of claim 1, wherein the pass-through device further comprises at least one data connector configured to operatively couple the generator to the surgical instrument for transmitting data signals therebetween.

9. The surgical system of claim 8, wherein the pass-through device further comprises a processor operatively coupled to the at least one optical component and to the at least one data connector, wherein the processor is configured to transmit control signals to the generator for adjusting the therapeutic energy based on data signals received by the processor from the at least one optical component.

10. The surgical system of claim 1, further comprising a spectrometer, wherein the at least one optical component is configured to operatively couple the spectrometer to the surgical instrument for transmitting light between the spectrometer and the at least one optical sensor.

11. The surgical system of claim 10, wherein the spectrometer includes a light source configured to transmit light to the at least one optical sensor and a light reader configured to receive light from the at least one optical sensor.

12. The surgical system of claim 1, wherein the pass-through device includes a pass-through box configured to be removably coupled to the surgical instrument and to the generator.

13. The surgical system of claim 1, further comprising a cable removably coupled to the surgical instrument and to the generator, wherein the pass-through device is presented by the cable.

14. The surgical system of claim 13, wherein the cable includes a distal cable plug removably coupled to the surgical instrument, wherein the pass-through device is presented by the distal cable plug.

15. The surgical system of claim 1, wherein the surgical instrument includes a proximal body and a distal body removably coupled to each other, wherein the shaft assembly extends distally from the distal body, wherein the pass-through device is presented by the proximal body.

16. A pass-through device for a surgical system, comprising:
(a) a housing;
(b) a first coupling member affixed to the housing, the first coupling member including at least one first therapeutic energy connector configured to receive therapeutic energy from a generator; and
(c) a second coupling member affixed to the housing, the second coupling member including:
(i) at least one second therapeutic energy connector operatively coupled to the at least one first therapeutic energy connector to receive the therapeutic energy from the at least one first therapeutic energy connector, and configured to transmit the therapeutic energy to an end effector of a surgical instrument, and
(ii) at least one optical fiber connector configured to transmit light to at least one optical sensor of the end effector and to receive light from the at least one optical sensor.

17. The pass-through device of claim 16, further comprising at least one optoelectronic component operatively coupled to the at least one optical fiber connector.

18. The pass-through device of claim 17, wherein the at least one optoelectronic component includes:
(i) a light source configured to transmit light to the at least one optical sensor, and
(ii) a light reader configured to receive light from the at least one optical sensor.

19. The pass-through device of claim 18, further comprising a processor operatively coupled to the light source and to the light reader, wherein the processor is configured to receive data signals from the light reader, wherein the processor is configured to send control signals to the light source.

20. A cable for a surgical system, comprising:
(a) a proximal cable plug; and
(b) a distal cable plug, wherein the distal cable plug includes:
(i) at least one therapeutic energy connector configured to transmit therapeutic energy between a generator and an end effector of a surgical instrument,
(ii) a light source configured to transmit light to at least one optical sensor of the end effector, and
(iii) a light reader configured to receive light from the at least one optical sensor.
